(19) United States
(12) Patent Application Publication
(10) Pub. No.: US 2014/0277308 A1
(43) Pub. Date: Sep. 18, 2014

(54) Adaptive Thermodynamic Therapy System

(71) Applicants: Raymond J. Cronise, Huntsville, AL (US); Anthony R. Kelley, Huntsville, AL (US); Scott Parazynski, Houston, TX (US); William D. Tepper, Alpharetta, GA (US)

(72) Inventors: Raymond J. Cronise, Huntsville, AL (US); Anthony R. Kelley, Huntsville, AL (US); Scott Parazynski, Houston, TX (US); William D. Tepper, Alpharetta, GA (US)

(21) Appl. No.: 14/216,157

(22) Filed: Mar. 17, 2014

Related U.S. Application Data
(60) Provisional application No. 61/802,992, filed on Mar. 18, 2013.

(51) Int. Cl. A61F 7/10

(52) U.S. Cl. CPC A61F 7/10
USPC 607/112

(57) ABSTRACT

An adaptive thermodynamic therapy system capable of comfortably increasing metabolic expenditure to facilitate excess weight loss, including one or more sensors for measuring a subject user's body temperature, current activity/metabolic level and providing data representative of said body temperature to a computer-based controller, and then actively controlling a thermal load in contact with subject user's body and responsive to the computer-based controller. In one embodiment, the controller is configured to receive input from at least one computer-based device configured to provide user body data and calculate a state value representative of the user body data and to adjust the thermal load to obtain a desired physiological response from the user by modifying the state values.

### CROSS-REFERENCE TO RELATED APPLICATIONS

This application claims priority to U.S. Provisional Application Ser. No. 61/802,992, entitled “Adaptive Thermodynamic Therapy System,” filed Mar. 18, 2013, and which is incorporated by reference as if fully set forth herein.

### DESCRIPTION OF THE PROBLEM

According to reports by the Center for Disease Control in 1990 no state had obesity prevalence equal to or greater than 15% (BMI) level. By 2009, only one State in the US remained below the 20% obesity level. In contrast, during that same year Americans spent an estimated $46 billion on diet products and self-help books. As the extent and severity of obesity grows in the United States, clearly another approach seems to be warranted. The current paradigm of weight loss thinking is focused on nutrition-exercise balance while thermodynamic factors, although critically important, are not even contemplated by current medical and diet industry.

It is well known that in homeotherms,¹ such as mammals and birds, a lifetime average temperature of 36-40° C. is maintained, but they live in an environment with average temperatures much lower. Most metabolic processes produce excess waste heat and this is used to maintain a core body temperature within +/−2° C. of individual set points. The described technology herein is directed toward a system for thermodynamically extracting heat from a person, which induces a corresponding increase in metabolism to maintain homeostasis.² The technology is able to extract heat energy from a user without discomfort, disruption of sleep or otherwise ¹ An organism that maintains its body temperature at a constant level, usually above that of the environment, by its metabolic activity.² The property of a system to regulate itself so that internal conditions remain generally constant. interfering with quality of life. In so doing, excess body mass can be reduced.

Similar efforts in the prior art include thermal blankets, liquid filled thermal vests, exercising in cool pools of water, space suit thermal control systems and other technology solutions. However, none have the ability to measure and adapt to a subject's comfort level or metabolic response while actively maximizing the calories consumed. Indeed, most cooling system devices fail because the subject's body adapts to the new environment and negates the desired benefits. For example, U.S. Pat. No. 4,718,429, to Smidt, have proposed cooling garments for fat loss, but no specificity is given on how to prevent the body's evolutionary survival instincts from reducing core temperatures to save energy on repeated cold or chilled exposure.

While there have been other attempts to cool the body in response to hyperthermia, in the treatment of central nervous system emergencies, surgical assistance, and even weight loss including Smidt, few have satisfactorily addressed the issues relating to responsive issues of hormesis³ and the complex non-linear response in the evolutionary drive to overall energy homeostasis. As such, prior devices designed towards weight loss through vest undergarments, cooling pads, hand mittens, etc. lack a core technology presented here. It is well known that the human body is extremely adaptive in nature and this evolutionary survival mechanism accounts for our ability to live in a wide range of thermal environments. Some of these adaptations are the ability to adjust our layers with clothing when appropriate. Still others involve building structures that can control the environment around us. These are obvious and easily recognizable adaptations to various external thermal conditions.

What is not as visible, or well understood, is the human body's ability to adapt at the metabolic level. Through a strong adaptive response ³ A generally favorable biological response to stressors, such as toxins, physical exertion, or thermal loads. to various external stimuli, the body can choose to habituate through regionally shunting blood flow and other mechanisms, essentially ignoring the external stimulus, or it can adapt by changing the resting metabolic rate. The latter adaptation can come in the form of an elevated heat production through central nervous system stimulation or through an equal suppression of average core body temperature. The body has the ability through regular exposure to respond in either one of these manners. Reducing the core temperature by a degree or two has been demonstrated in several different cultures of deep-sea free divers and natives that sleep uncovered at low temperature. Unfortunately, this type of adaptive response leads to lower resting metabolic rate requirements that limit long-term weight loss.

It is known that sirtuins function to maintain homeostasis and secure an organism's survival when exposed to internal and/or external perturbations. SIRT1, in particular, is a key regulator of energy homeostasis and energy metabolism via the deacetylation of PGC-1α, a transcriptional co-activator. SIRT1 and PGC-1α are critical mediators of mitochondrial biogenesis and may be responsible for many of the health benefits of exercise. Interestingly, the other main function of PGC-1α is another survival trait, non-shivering thermogenesis, during which individual mitochondrion are able to bypass ATP production and instead create heat through the activation of uncoupling protein, UCP-1. This is a very efficient mechanism to replace the immediate response to cold, shivering, which leads to exhaustion and potential muscle damage, with a mechanism that produces heat directly through the recruitment of mitochondria.

Consequently, when the body is exposed to lower temperatures, it also has the ability to maintain core temperature and thereby increase metabolic output to match increased losses to the exposed external temperature. This desired increase in metabolic output can lead to increased daily caloric requirement. When combined with a fixed caloric intake, this leads to caloric deficit and subsequent weight loss.

In order to overcome the body's natural adaptive mechanisms to a cold challenge, a novel approach involving a dynamic, closed-feedback system is required to maximize thermogenic caloric expenditure while at the same time no becoming physically uncomfortable, impacting the quality of one's sleep or being nullified by physiologic habituation.

At the same time, our current society is also one that is chronically sleep deprived. Links between sleep and metabolic dysfunction can be found in early Roman medicine, and too little sleep has more recently been associated with obesity and type 2 diabetes, as well as stroke, coronary heart disease, hypertension, respiratory disorders, and poor self-rated health. Early observations ranging from Australian aborigines to the cold climate of the Scandinavian Nomadic Lapps demonstrate how adaptable humans are to mild cold stress during sleep. Adaptation also occurs for non-native inhabitants after repeated cold stress exposure. Furthermore, until the 20^{th} century, winter was characterized by long nights without artificial light and generally cooler sleeping conditions.

Much of the same biology that allows winter adaptation for cooler environments, including sleep, overlap the underlying metabolic mechanisms involved in adaptations to caloric scarcity. Increased sleep in cool environments and long nights of winter in the absence of excess artificial light and warmth may also work synergistically on our metabolism, promoting the conservation of valuable calories in a time of year when they are naturally scarce. Furthermore, melatonin, a hormone associated with sleep synthesized by the pineal gland from serotonin, induces the body to sleep by lowering core body temperature. In fact, a steep rate of decline in core body temperature is associated with both the onset and quality of sleep.

In contrast, reduced sleep appears to lead to impaired glucose tolerance and increased insulin resistance, increased appetite through changes in leptin and ghrelin levels, and reduced energy expenditure. One might conceptually associate winter's cold, dark, and still environment as a natural balance to summer's warm, bright and active environment. Moreover, the social norms of heavy blanketing began in times when bedrooms were rarely heated. Very few of us now sleep in the cold, and studies have even shown an association between weight gain and average room temperature.

These ritual habits in sleeping condition aren't changed easily. For example, many people have the desire to enter a warm bed, even though a slight drop in body temperature is required for sleep. However, people generally rest and sleep more soundly in a cooler environment. The system described below may be configured to create both a warm environment for going to sleep or waking, but rapidly change to a cooler state once the onset of sleep is detected.

Accordingly, the system set forth below provides a therapeutic application of a thermal load to increase metabolism, but solves the problem of the body's natural adaptation responses to such thermal load. In addition, the system may be adapted to improve sleep quality.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 is a functional diagram of an exemplary adaptive thermodynamic therapy system;

FIG. 2 is a functional diagram of another exemplary embodiment of an adaptive thermodynamic therapy system within an enclosed chamber or room;

FIG. 3 is a functional diagram of another exemplary embodiment of an adaptive thermodynamic therapy system within a water bath;

FIG. 4 is a functional diagram of a control processor suitable for achieving the various objectives of the embodiments described in connection with FIGS. 1 through 3;

FIG. 5 is a functional diagram of a further exemplary embodiment of an adaptive thermodynamic therapy system, showing overall data flow;

FIG. 6 is a process diagram of some of the functions performed by the exemplary embodiment of the system described with reference to FIG. 5;

FIG. 7 is a relational network architecture in which an adaptive thermodynamic therapy system may operate;

FIG. 8 is a functional diagram of an exemplary adaptive controller; and

FIG. 9 presents various exemplary embodiments of thermal loads.

### DETAILED DESCRIPTION

The various embodiments of the present invention and their advantages are best understood by referring to FIGS. 1 through 9 of the drawings. The elements of the drawings are not necessarily to scale, emphasis instead being placed upon clearly illustrating the principles of the invention. Throughout the drawings, like numerals are used for like and corresponding parts of the various drawings.

Furthermore, reference in the specification to “an embodiment,” “one embodiment,” “various embodiments,” or any variant thereof means that a particular feature or aspect of the invention described in conjunction with the particular embodiment is included in at least one embodiment of the present invention. Thus, the appearance of the phrases “in one embodiment,” “in another embodiment,” or variations thereof in various places throughout the specification are not necessarily all referring to its respective embodiment.

It is proposed in the present disclosure that the randomization of warm and cool exposure during various sleep states, or while seated at one's desk at work, can effectively mitigate lower body temperature adaptation and advantageously place the body in a state of increased resting metabolic rate. Additionally, and of equal importance, modification of the user's thermal environment can be applied in a manner to encourage sound sleep, improve sleep quality, and maximize comfort. Due to the rapid, automated adaptability of the proposed technology additional benefits are achieved such as the ability to actively mitigate hot flashes brought on during menopause or induced by medications, provide comfort during febrile illness, aid in the ability to increase effectiveness of metabolic drug therapies, and the ability to stimulate human sirtuin levels to increase life span.

Additionally, the device can be used while awake to enhance alertness and increase caloric consumption leading to better weight loss gains while exercising, sitting at your desk, or performing other tasks. In sum, our Adaptive Thermodynamic Therapy System is a closed-loop cooling system that monitors and adapts to one's unique physiology, accelerating metabolism and thus adipose weight reduction. This is accomplished by means of a uniquely adaptive control system with integrated hardware and software as described in this disclosure.

Each person will have a unique signature of sleeping patterns, REM and NREM⁴, along with a similar unique lower temperature level comfort of sleeping. By employing adaptive systems, an individual plan can be learned from the user's sleeping habits and the maximum amount of caloric energy can be withdrawn during each sleep session. This approach is completely unique as no other system can adaptively learn or is aimed at overcoming the evolutionary limits of habituation and adaptation.

Similar arguments can be made for exercise systems in an aqueous environment. Water has over a twenty-fold increase of thermal conductivity than air. As such, a person submerged in a water bath that is thermally controlled using the same adaptive system approach can be fine-tuned to minimize adaptation and maximize participant comfort and thermal loading. This system, combined with physical exertion, can lead to an increased caloric deficit of the body relative to the exercise alone.

With these concepts in mind and with reference now to FIG. 1, an exemplary adaptive thermodynamic therapy system 100 includes a computer-based control processor 101 configured with control logic (described in detail below) that, when executed, performs control functions described herein based on sensor data input 102a-c. Such input may be from environmental sensors, for example, a sound monitor 103a, and a plurality of temperature sensors 103b. Other examples might include ambient light detectors, ambient temperature, regional airflow and O2/CO2 sensors. Sound monitor 103a may be used detect room noises such as snoring, talking in sleep, periodic sleep disturbing sounds, etc. to help improve sleep quality and identify conditions such as sleep apnea and other sleep disorders. Accordingly, control processor 101 is preferably configured with control logic that compares data 102a input from the sound monitor to a database of sound profiles and outputs a result corresponding to identification of sound input from the monitor 103a. The system might also record peak levels for later analysis and isolation.

In addition, the controller 101 preferably receives data 108 from a sleep monitoring system 109 that monitors quality and sleep state of the user. Sleep state monitoring systems are available commercially as smartphone applications, for example, the Zeo™ Sleep Manager, offered by Zeo, Inc., and Up, offered by Jawbone®. Another source of input is user data 117, which may comprise body composition data 123 (e.g., weight, height, body mass index (BMI), body fat percentage, among other data), body vital measurements 125 e.g., pulse, respiratory rate, VO₂, temperature, thermal transfer rates, motion monitoring accelerometers and other biometric information. In addition, exhaled ppO2 and ppCO2 nasal sensors could also be employed as an input to this closed-feedback loop system. The system may also obtain manually entered subjective assessment data 127 (e.g., quality of sleep, physical sensation, etc.), input by the user through any suitably configured computer-based device, including a desktop or laptop personal computer, or a smartphone or worn computer.

Control functions performed by the processor 101 may include adjustment of light, sound and atmospheric temperature within the user's environment by coupling command signals to light and sound controls 105, and temperature controls 107. A white noise generator might also be employed to blunt environmental noise if detected above a predetermined threshold.

The illustrated embodiment 100 also includes a thermal load device 111 that is essentially a heat exchange apparatus in thermally conductive contact with a user body. As illustrated in FIG. 9, the thermal load device 111 could be, without limitation, a bed pad 901, boots 903, vest 905, blanket 907, head gear 909, chair pad 911, an arm wrap 913, a leg wrap 915, gloves 917 or pants 919. In one embodiment, the thermal load device 111 is a temperature-controlled bed 915. In an alternate embodiment, the thermal load device 111 is a head worn device 909 with integrated fluid circuits to distribute cooled water as directed by the processor 101. A head worn device 909 has the advantage of having minimal impact on mobility during sleep, as the user can roll from side to side without tugging substantially on the chilled fluid line. The thermal load device 111 preferably comprises sensors 129 to measure user temperatures 129a, perhaps at a variety of locations on or in the user's body, heat transfer to/from the user's body 129b, user's body motion 129d and the caloric load 129c experienced by the user during sleep. Data from the sensors 129 are transmitted to the control processor 101 for establishing a control loop for the adjusting the temperature of the unit in response to user data and/or as part of a timed profile. In one embodiment, data 110 from the thermal sleep unit 101 sensors 129 may be transmitted to the control processor 101 which is configured with control logic that compares the data 110 to a database of temperature and heat transfer profiles and determines whether the user is experiencing a “hot flash,” such as caused by menopause or medications. Based upon this determination, the control processor transmits a control signal 112 to a thermal unit temperature regulator 113 that heats or cools the thermal load device 111 as required by the control processor 101.

Optionally, control processor 101 may be in communication with a distributed data network 121 (e.g., LAN, WAN, Internet, or the like) via a network server 119. In this way, user data may be transmitted to a network informational site for system performance tracking, data mining, and control optimization across multiple users. Moreover, the information site may comprise a user forum for obtaining user feedback, sleep analysis, and exchanging user information. In addition, control processor 101 may be configured to retrieve data from an identified user, or multiple users to define or adjust temperature profiles to meet user's unique goals, desires, and comfort. Anonymized data can also be collected and archived from a large number of users for later analysis and improvement of the control algorithm. Additionally, multiple users can also share their results in an online community, sharing experiences, competing for best results and providing mutual encouragement. (“gamification” and social media integration)

Another exemplary embodiment is illustrated in FIG. 2 wherein a user is placed inside an enclosed chamber or small room 201. Control processor 101 may provide control of environmental parameters of the chamber 201 e.g., light, sound, through control signals 104. To this end, optionally, environmental sensors 103 may provide measurement data 102 to the control processor 101 as in the previously described embodiment. (See FIG. 1). This embodiment preferably includes gas sensors 203 configured to measure the amounts of, for example, oxygen and carbon dioxide. This gas measurement data 206 is provided as input to the control processor 101 which may be configured with control logic to determine a user's metabolic rate.

The chamber 201 is configured with temperature adjustment control 205 with a temperature sensor 207 that measures the temperature within the chamber 201. A temperature data signal 208 is provided to the control processor 101 which is configured with control logic to compare the temperature data 208 to a pre-defined threshold, which may be defined as part of a timed profile or in response to changes in the user's body temperature, and determines whether to issue a control signal 210 to adjust the temperature to maintain profile temperature.

Similarly, FIG. 3 presents a further exemplary embodiment wherein a user is placed inside a water bath 301, or is enclosed in a water-filled heat exchange apparatus in thermally conductive proximity to the user's body. Similar to the previous embodiment, the water bath 301 is configured with temperature adjustment control 305 with a temperature sensor 307 that measures the water temperature within the bath 301. A water temperature data signal 308 is provided to the control processor 101 which is configured with control logic to compare the water temperature data 308 to a pre-defined threshold, which may be defined as part of a timed profile or in response to changes in the user's body temperature, and determines whether to issue a control signal 310 to adjust the water temperature to maintain profile temperature.

The control processor 101, as will be appreciated by those skilled in the arts, may be one or more computer-based processors 101. Such a processor may be implemented by a field programmable gated array (FPGA), application specific integrated chip (ASIC), programmable circuit board (PCB), other suitable integrated chip (IC) device or other suitable electronic monitoring and control circuits.

With reference to FIG. 4, a processor 101 in effect comprises a computer system. Processor 101 may consist of, or comprise, one or more general-purpose processing devices 401 such as a microprocessor, central processing unit, or the like, coupled to a communication bus 403. More particularly, the processor 101 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 101 may also be one or more special-purpose processing devices such as an application specific integrated circuit (ASIC), a field programmable gate array (FPGA), a digital signal processor (DSP), network processor, or the like. Processor 101 is configured to execute the control logic 422 for performing the operations and steps discussed herein. The processor 101 can also include a main memory 405, such as, without limitation, flash memory, read-only memory (ROM), or random access memory (RAM), and can also include a secondary memory 407 that includes a machine-readable storage medium 413 having stored therein computer software and/or data. While the machine-readable storage medium 413 is shown in an exemplary embodiment to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present invention. The term “machine-readable storage medium” shall accordingly be taken to include, but not be limited to, solid-state memories, and optical and magnetic media.

Control logic 422 (also called control logic 422 or software) is stored in the main memory 405 and/or secondary memory 407. Control logic 422 can also be received via the communications bus 403 from an input device 407. The control logic 422 allows for adaptive learning relative to a given user; the cool exposure planning algorithm is iterative, based on REM sleep duration, overall sleep quality metrics, fatigue assessments and trends in weight loss. Such control logic 422, when executed, enable the processor to perform certain features as discussed herein.

A processor 101 may advantageously contain control logic 422 or other substrate configuration representing data and instructions, which cause the processor 101 to operate in a specific and predefined manner as, described hereinabove. The control logic 422 may advantageously be implemented as one or more modules. The modules may advantageously be configured to reside on the processor memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro-code, circuitry, data, and the like. In programmable logic circuits, such as FPGAs, ASICs, Neural Net chips, etc., control logic can be partially or fully hardwired into functional circuits. Control logic 422 may be installed on the memory using a computer interface 411 couple to the communication bus 403 which may be any suitable input/output device. The computer interface 411 may also be configured to allow a user to vary the control logic 422, either according to pre-configured variations or customizably.

In addition, the processor 101 may include a network interface 409. Thus, control logic 422 may further be transmitted or received over a network 520 via the network interface device 508.

It should also be understood that the programs, modules, processes, methods, and the like, described herein are but an exemplary implementation and are not related, or limited, to any particular processor, apparatus, or processor language. Rather, various types of general purpose computing machines or devices may be used with programs constructed in accordance with the teachings described herein. Similarly, it may prove advantageous to construct a specialized apparatus to perform the method steps described herein by way of dedicated processor systems with hardwired logic or programs stored in nonvolatile memory, such as, by way of example, read-only memory (ROM), for example, components such as ASICs, FPGAs, PCBs, microcontrollers, or multi-chip modules (MCMs). Implementation of the hardware state machine so as to perform the functions described herein will be apparent to persons skilled in the relevant art(s).

FIG. 5 illustrates an example of data flow executed by the adaptive thermodynamic therapy system. The adaptive controller 501 may be the control logic 422 executed by the control processor 101. The adaptive controller 501 receives data from a number of sources, for example, and without limitation, user environment and activity data 515, user food/nutrient intake data 513, user medical therapy agents data 511, sleep quality data 509, direct metabolic measurement data 507 and physiological biomarkers data 505. Adaptive controller 501 creates a multi-dimensional characterization of the user based upon these data, records such characterization in a database, and generates a user-specific state equation that expresses the user's current multi-dimensional state vis-á-vis the recorded data. The adaptive controller 501 then performs a cluster analysis upon the user-specific state equation, comparing it to clusters of other user-specific records to assess what behavior modifications may be required for the user to adopt for the user to reach the desired physiological state, and to determine adaptive application of cool to cold stress 503 to the user. Data may be manually entered or provided by appropriate sensors.

Exemplary user environment data 515 includes room temperature, outside weather data and user activity such as exercise or housework. Temperature and weather data may be obtained from environmental sensors having connections to a network, including a local network, or the Internet. Activity data may be manually entered or may be obtained from a wearable device that records the user's movements, e.g., an activity bracelet or other worn accelerometers. Non-limiting examples of suitable activity-recording bracelets may be found in products such as Jawbone® UP™, Nike+Fuelband®, Fitbit®, and Amiigo™. Activity may also be monitored via video monitoring. In addition, a smart phone may be configured with an application for recording the data, either manually entered by the user or through networked connection with appropriate sensors, and transmitting the data 520 to the adaptive controller 501.

Similarly, data representing the user's food intake 513 may be manually entered via any computer with an appropriate user interface, e.g., a smart phone configured with a suitable application, and may record such data as macro- and micro-nutrient information and calories, including any dietary supplements. Such data may be provided by integrating data from meal plans that list the nutritional information of food consumed according to such a plan.

Manually entered medical therapy data 511 may include, prescription and non-prescription drugs, hormone therapy, brown adipose tissue manipulation, and any other stimulants, depressants or sedatives. Other variables such as weight, resting heart rate, blood pressure, physical exercise performed, etc. may also be manually entered.

Sleep quality data 509 measures the duration of a user's sleep period, whether the user's sleep is light or deep, the number of times a user may wake during sleep periods, the degree of user bodily activity during sleep as well as the user's basal metabolic rate. Additionally, these data could include the degree of body temperature change during sleep. These data would necessarily need to be obtained through a variety of sensors with which a bed is equipped. Such sensors include, for example, temperature sensors, accelerometers, ambient light, ambient sound, and O₂ and CO₂ sensors. The latter sensors may also be used to provide direct metabolic measurement data 507, which includes, for example, CO₂ produced, O₂ consumed, and from which a respiratory quotient may be derived along with the user's resting metabolic rate and resting energy expended. In addition, an embodiment of the system could be configured to measure the user's rapid-eye-movement (REM) sleep. Methods for measuring REM sleep include, in addition to measuring body movement via the afore-mentioned accelerometers, electroencephalography, polysomnography, and eye movement monitors. Smart phone applications listed above may also be employed to help register REM sleep periods. Post-sleep alertness testing on smart phone applications could also be used as an independent assessment of overall sleep quality and wakefulness. Such applications are now commercially available, but their output would be fed back into the system's control algorithm.

The last data category, physiological biomarker data 505, comprises blood pressure data, blood glucose data, cholesterol levels, hemoglobin A1C data, user body fat, body and skin temperature, perspiration, heart rate, respiratory rate, skin conductance and oxygen saturation. Such data may be manually entered or obtained automatically through appropriate sensors. Other transcutaneous, blood and saliva-based biomarkers of sleep, wakefulness and hormonal control might eventually be measured and fed into the control system.

With reference now to FIG. 6, an exemplary process performed by the adaptive controller will be described. Initially, the adaptive controller 501 obtains user-input data 601, user sleep data 602, user metabolism data 603, and user physiological data 604 as described above. Next, using these data, the adaptive controller uses the data to generate a multi-dimensional state equation 605 which incorporates each item of data as a dimension and expresses the current state of the user. This current state is then compared, at step 606, with a cluster defined in the multi-dimensional space of users with similar multi-dimensional states but that represent a cluster that includes the goal state of the current user, or a “desired cluster.” At 607, the adaptive controller 501 evaluates whether the values of the user's current dimensions are within those defined by the desired cluster. If so, the system does not engage in modification of the user's environment 609. If the values of the current user's dimension are not within those of the desired cluster, the system modifies the user's physiological response systems by modifying the user's environment through subjecting the user to cool stress 608, 611 as described previously. In addition, in other embodiments, the system may suggest voluntary behavioral modifications 610 by providing feedback 512, 516, 520 in the areas of medical therapies 511, nutritional consumption 513, and user environment and activity 515. Such could be displayed on one's laptop, smartphone, worn computer or any other connected display device.

In essence, the system is configured to manipulate the user's physiological response to a cold load in order to affect one or more of the user's dimensions, i.e., improve sleep quality, stimulate weight loss/increase metabolism, mitigate menopausal hot flashes, treat or palliate febrile illness, improve health, increase life span, increase alertness, and/or increase the amount of user brown adipose tissue. The true advantage of this system and technique relates to the human body's tendency to adapt to its environment. For example, the user's body may come to tolerate the temperature and timing the cold stress application reducing the effectiveness of the therapy. However, since the system constantly measures the user's multi-dimensional state versus the user's physiological ideal state, if the current state is still outside the ideal state, the system will change the timing and/or the temperature rate of the cold stress application, tricking the body's response system.

Further, the system may be configured to energize the cold stress application device randomly, thereby reducing the likelihood that a user's body will adapt to timing of cold stress application. The adaptive controller 501 is preferably configured to be responsive to the various sensors listed above, and monitor the user's body temperature responses to cold stress for subtle changes in fractions of degrees. When the body core temperature starts adjusting, i.e., decreasing in cold response, the control system warms the body. Any time the body starts such adaptation, the control system modifies conditions to counter the body response. Once somewhat steady state results are reached, the adaptive controller 501 may be configured to randomly and temporarily alter different parameters such as cool down rate, final cold temp, time at given temperature, warm-up rate, light, sound, etc., and to monitor for additional benefits. If the body responds in a desired way to a given stimuli, this stimuli will be added into the control algorithm as long as it provides beneficial responses.

In a variant embodiment, regional variation in cold and warm thermal application might be used to maximize thermogenic caloric expenditure while minimizing blood shunting, sleep disruption and whole body thermal discomfort. This could be accomplished by use of multiple, independently controlled thermal application pads, e.g., pads in thermal contact with the scalp, torso, abdomen, and extremities.

In yet another embodiment, adaptive controllers 501 for a plurality of users may be configured to share data collected from respective users through access to the data network 121. As such, data findings may be shared with other user adaptive controllers 501 to monitor, test, and induce the desired beneficial body responses of other users. In this way, a given individual or common cluster group can be continually probed for additional stimuli that may help them move to a more desirable state. The methods employed to create the random stimulus are statistically based and applied to groups via genetic algorithm manipulation. The individual and group responses are evaluated by artificial intelligence (neural nets, fuzzy logic, etc.) and statistical methods to determine stimulus modification reactions and to strengthen desired stimulus while eliminating or weakening stimulus that doesn't help a given user reach their desired goal. Each user will have a customized, unique control algorithm at any given time as created by artificial intelligence algorithms.

With reference again to FIG. 9, yet another embodiment of the adaptive thermal therapy system includes at least one of a variety of thermal load devices, each of which are thermally regulated by a thermal unit temperature regulator 113 controlled by a control processor 101. In addition, however, thermal load devices may be configured with a plurality of cooling zones each separately controlled and initiated by the control processor 101. For example, a bed 901, which may also be a bed pad, may include zones 901a-c. Chair pad 911 could include zones 911a, 911b. Further, a plurality of the garments, i.e., the head gear 909, the vest 905, the arm wrap 913, the gloves 913, the pants 919, the leg wraps 915, or the boots 903, may be worn concurrently and all separately controlled by control processor 101. Accordingly, another manner in which the system may overcome any physiological adaptation a subject user's body manifests is by changing the area of the subject user's body that is subjected to the thermal load. In other words, any of the bed zones 901a, 901b, 901c may be activated to provide cooling to the corresponding area of the user's body in thermal contact with such zones. Likewise, any of the garments may be activated to cool the portion of the body upon which the garment is worn.

Further, a bed 901, pillow 921 and/or blanket 907 as the thermal load in this system may provide additional therapeutic benefits toward a user's sleep quality. It is well-known that there is a relationship between body temperature and sleep state. Body temperature decreases during synchronized, slow wave sleep (SSWS), or non-rapid eye movement (NREM) sleep, and increases during periods of desynchronized, paradoxical sleep, or rapid eye movement (REM) sleep. Moreover, sleep studies indicate that optimal environmental temperature for sleep is around 60 to 68° F. Most users, however, would prefer to enter a warm bed prior to sleep.

Therefore, according to an embodiment of the present system, the control processor 101 may regulate a cooling bed 901 or blanket 907 such that the user gets into bed when the bed/blankets are at a user-preferred temperature that may be pre-set by the user. During REM sleep, as indicated by sleep quality data, bed temperature will be held at the pre-set value or at some average temperature. Then, when the control processor detects user sleep quality data 509 indicating the user is entering NREM sleep, the control processor 101 may initiate a decrease in the bed temperature. Bed/blanket temperature may be modulated throughout the sleep period based upon data indicating the user's entry into different sleep phases. When the sleep quality data 509 indicates the user is about to awaken, the control processor 101 may be configured to initiate an increase in bed temperature to assist in the waking process. Alternatively, the control processor may be configured to modulate the bed/blanket temperature based simply on time, for example, cooling the user after some period of time and then warming some period of time prior to waking. Thus, the system may help a user fall asleep, improve quality of his or her sleep, and help the user wake by monitoring sleep stages and adjusting thermal environment to optimal levels during each sleep stage.

In accordance with another exemplary embodiment of the system, FIG. 7 illustrates a networked computing system 700 including various wireline and wireless computing devices that may be utilized to implement any of the sensor data acquisition, data transmission and system control processes associated with various features of the system set forth herein. The networked computing system 700 may include, but is not limited to, a group of remote server devices 704a-c, any one of which may be associated with various adaptive controllers 501 in an embodiment in which an adaptive controller 501 is based remotely from the user; a data communications network 121 (including both Wide Area Network (WAN) and Local Area Network (LAN) portions); a variety of remote wireless communications devices, including cellular phones 708a-b and Personal Data Assistant (PDA) devices 710, that may be connected to the data communications network 121 utilizing one or more wireless base station 706 or any other common wireless or wireline network communications technology; one or more network gateway or switch devices 712 that can facilitate data communications processes within the LAN and between the LAN and the WAN of the data communications network 702; a desktop computer 720; a wireless router 722 that may communicate with various wireless LAN devices using any common local wireless communications technology, such as Wi-Fi™ or unshielded twisted pair cable; a wireless laptop computer 724; a tablet computer 726, a wireless system control unit 728 in an embodiment in which the adaptive controller 501 is co-located with the user; and a cellular phone device 730. In addition, the various sensors 732 and environmental or cold stress application system controls 734, 736 described above may be in wireless or wireline communication via the LAN.

FIG. 8 shows a block diagram view of an exemplary adaptive controller 501. The adaptive controller 501 may include, but is not limited to, one or more processor devices including a central processing unit (CPU) 802. In an embodiment, the CPU 802 may include an arithmetic logic unit (ALU, not shown) that performs arithmetic and logical operations and one or more control units (CUs, not shown) that extract instructions and stored content from memory and then executes and/or processes them, calling on the ALU when necessary during program execution. The CPU 802 is responsible for executing all computer programs stored on the adaptive controller's volatile (RAM) 812 and nonvolatile (ROM) 810 which includes, as set forth, above the adaptive controller's user data analysis and the control response processes. The adaptive controller 501 may also include, but is not limited to, a user interface 806 that allows a user to interact with the adaptive controller's software and hardware resources; a database 808 that includes user data, either data associated with an individual user or with multiple users; a transceiver 820 for transmitting and receiving data over the data communication network 802; and a system bus 822 that facilitates data communications amongst all the hardware resources of the adaptive controller 501.

As described above and shown in the associated drawings, the present invention comprises an adaptive thermodynamic therapy system and method. While particular embodiments have been described, it will be understood, however, that any embodied invention is not limited thereto, since modifications may be made by those skilled in the art, particularly in light of the foregoing teachings. It is, therefore, contemplated by the appended claims to cover any such modifications that incorporate those features or those improvements that embody the spirit and scope of the system.

What is claimed is:

1. An adaptive thermodynamic therapy system comprising:
a plurality of sensors for measuring a subject user's physical state, said physical state being defined by a plurality of parameters, said parameters being at least two of user metabolism, user physiology, user nutrition, user activity, user environment, and user sleep quality, and then providing multi-dimensional data representative of said physical state;
a computer-based controller configured with a memory comprising control logic that causes said controller to perform a process comprising the steps of:
i. receiving said multi-dimensional data;
ii. determine a subject user state based upon said multi-dimensional data;
iii. comparing said subject user state to an ideal state; and
iv. causing application of a thermal load to said subject user's body for optimizing cool stress to increase said subject user's metabolic rate if said subject user state does not correspond with said ideal state; and
a thermal load in contact with said subject user's body and responsive to said computer-based controller.

2. The adaptive thermodynamic therapy system of claim 1, further comprising a sleep monitor for monitoring the quality and duration of a user's sleep and configured to provide data representative of said quality and said duration to said controller.

3. The adaptive thermodynamic therapy system of claim 1, integrated into a desk chair in such a way to deliver cool stress, accelerate metabolic workload to shed adipose weight, and to simultaneously increase alertness and productivity.

4. The adaptive thermodynamic therapy system of claim 1, further comprising a device configured to provide to said controller user body data, said user body data being at least one of body composition data, physiological data, respiration data, metabolic data, subjective assessment data, activity data, medicament data and nutritional data.

5. The adaptive thermodynamic therapy system of claim 4, further comprising environmental sensors for providing data representative of said subject user's environment to said controller.

6. The adaptive thermodynamic therapy system of claim 5, wherein said environmental sensors are at least one of light sensor, a temperature sensor, a sound sensor, an oxygen sensor and a carbon dioxide sensor.

7. The adaptive thermodynamic therapy system of claim 1, wherein said thermal load is at least one of:
a temperature-adjustable bed,
a temperature-adjustable bath,
an abdominal pad or garment
a buttocks and thighs-worn garment
a temperature adjusted body suit,
a temperature-adjustable vest,
a temperature-adjustable glove,
a temperature-adjusted leg wrap,
a temperature-adjusted arm wrap,
a temperature-adjusted foot wrap,
a temperature-adjusted head wrap,
a temperature-adjustable blanket,
a temperature-adjustable pillow, and
a temperature-adjustable chair.

8. The adaptive thermodynamic therapy system of claim 7, wherein said thermal load comprises a plurality of zones, each said zone being in thermal contact with a different region of said subject user's body, and wherein said controller is configured to change the zone through which said thermal load is applied.

9. The adaptive thermodynamic therapy system of claim 1, further comprising a network interface for communicating with a computer-based data network.

10. The adaptive thermodynamic therapy system of claim 9, wherein said process further comprises the step of:
assessing the rate of temperature decline that is most optimal for inducing sleep in said subject user; and
wherein said rate of temperature decline is a dimension of said multi-dimensional data.

11. The adaptive thermodynamic therapy system of claim 9, wherein said process further comprises the steps of:
obtaining a plurality of subject user states representative of a plurality of other users through said network interface;
comparing said subject user state to said plurality of states;
calculating a user ideal state based upon a result of said step of comparing; and
adjusting said thermal load relative to said subject user's body to affect a physiological response from said user causing a change of one or more dimensions of said plurality of dimensions with respect to said ideal state.

12. The adaptive thermodynamic therapy system of claim 1, wherein said process further comprises the steps of:
comparing said user body data to earlier data received in relation to said user;
determining whether said user's body has become unresponsive to current adjustments to the thermal load, said adjustments being at least one of timing adjustment, duration adjustment and temperature adjustment; and
modifying at least one of said timing adjustment, duration adjustment and temperature adjustment.

13. The adaptive thermodynamic therapy system of claim 12, further comprising a network interface for communicating with a computer-based data network.

14. The adaptive thermodynamic therapy system of claim 13, wherein said process further comprises the steps of:
obtaining a plurality of state values representative of other users through said network interface;
comparing said user state value to said plurality of state values;
calculating a user ideal state value based upon a result of said step of comparing; and
adjusting said thermal load relative to said subject user's body to affect a physiological response from said user causing a change of one or more dimensions of said plurality of dimensions with respect to said ideal state value.

15. A method for affecting a physiological change in a subject user using an adaptive thermodynamic therapy system, said method comprising the steps of:
obtaining user input data, said user input data comprising at least one of user environmental data, user activity data, user nutritional data, user medicament data, and user body state goals data;
obtaining user sleep data, said user sleep data comprising at least one of sleep quality and sleep duration;
obtaining user metabolic data;
obtaining user physiological data, said physiological data comprising at least one of:
i. body fat percentage;
ii. body temperature;
iii. respiratory rate;
iv. oxygen saturation;
v. heart rate;
vi. skin conductance, and
vii. blood data;
generating a multi-dimensional state value representative of said user input data, said sleep data, said metabolic data and said physiological data;
comparing said state value against a group of state values corresponding to a plurality of other users; and
applying a cold stress to said subject user based upon a result of said step of comparing.

16. The method of claim 15, further comprising the steps of:
determining a user physiological response to said step of applying a cold stress based upon said user input data, said user sleep data, said user metabolic data and said user physiological data; and
modifying application of said cold stress by changing at least one of timing, duration and temperature of said application.

17. An adaptive thermodynamic therapy system comprising:
one or more sensors configured to obtain measurements indicating user sleep quality and to convert said measurements into data representative of user sleep quality; and
a control processor responsive to said one or more sensors and configured with a memory comprising control logic that causes said control processor to perform a process comprising the steps of:
i. receiving said user sleep quality data;
ii. determining a user sleep state based upon said data, said sleep state being one of REM and NREM; and
iii. initiating cooling through a selectively initiatable thermal load in the event said sleep state is NREM, said thermal load being at least one of a cooling bed, a cooling pillow, and a cooling blanket.

18. An thermodynamic therapy system comprising:
at least one of a bed, a pillow, and a blanket, each of which being configured to be temperature-adjustable and responsive to a control processor configured with a memory having control logic that causes said control processor to modulate the temperature of said bed, pillow or blanket based upon at least one of the user's sleep state or time.
